(19) United States
(12) Patent Application Publication
(10) Pub. No.: US 2004/0116411 A1
(43) Pub. Date: Jun. 17, 2004

(54) Combination HIV therapy including camptothecin

(75) Inventors: Gerald Schochetman, Libertyville, IL (US); Lucy Chang, San Mateo, CA (US); Joseph Rubinfeld, Danville, CA (US)

Correspondence Address: WILSON SONSINI GOODRICH & ROSATI, 650 PAGE MILL ROAD, PALO ALTO, CA, 943041050

(73) Assignee: SuperGen, Inc., a Delaware Corporation, Dublin, CA

(21) Appl. No.: 10/670,179

(22) Filed: Sep. 23, 2003

Related U.S. Application Data
(63) Continuation of application No. 09/606,967, filed on Jun. 28, 2000, now abandoned.

(51) Int. Cl. A61K 31/551; A61K 31/4745

(52) U.S. Cl. 514/220; 514/283

(57) ABSTRACT

A method is provided for treating HIV infection using a combination therapy which includes a compound selected from the group consisting of 20(S)-camptothecin, analog of 20(S)-camptothecin, derivative of 20(S)-camptothecin, precursor of 20(S)-camptothecin and metabolite of 20(S)-camptothecin, in combination with a cocktail of antiretroviral drugs such as nucleoside reverse transcriptase inhibitors, non-nucleoside HIV reverse transcriptase inhibitors, protease inhibitors, fusion inhibitors and integrase inhibitors. The method comprises administering highly active antiretroviral therapy (HAART); and co-administering to the HIV-infected host therapeutically effective amount of a compound selected from the group consisting of 20(S)-camptothecin, analog of 20(S)-camptothecin, derivative of 20(S)-camptothecin, precursor of 20(S)-camptothecin and metabolite of 20(S)-camptothecin.

### CROSS REFERENCE TO RELATED APPLICATION

This application is a continuation of U.S. application Ser. No. 09/606,967, filed Jun. 28, 2000, entitled “Combination HIV Therapy Including Camptothecin,” which is herein incorporated by reference.

### BACKGROUND OF THE INVENTION

1. Field of the Invention

This invention relates to compositions and methods for treating infectious viral diseases, and more particularly relates to combination therapy that includes camptothecin in the treatment of HIV infection and AIDS.

2. Description of Related Art

Human immunodeficiency virus (HIV) has been implicated as the primary cause of the slowly degenerate disease of the immune system termed acquired immune deficiency syndrome (AIDS). Infection of the CD4⁺ subclass of T-lymphocytes with the HIV type-1 virus (HIV-1) leads to depletion of this essential lymphocyte subclass which inevitably leads to opportunistic infections, neurological disease, neoplastic growth and eventual death.

Many antiviral drugs have been developed to inhibit HIV infection and replication by targeting HIV reverse transcriptase (e.g. AZT, ddI, ddC, d4T, 3TC) and proteases (RITONAVIR, INDINAVIR, and NELFINAVIR). Treatment following a prolonged single drug regimen has met with limited success where there is a relatively small drop in viral load, followed by a rise in the amount of detectable virus in blood, presumably due to the development of drug resistance strains of HIV. The resistance of HIV to drugs is not only associated with the high mutation rates of HIV but also due to the selective pressure of prolonged anti-HIV drug therapy. It has also been demonstrated that the emergence of drug resistance in HIV-1 correlates with the presence of point mutations in the targeted protein. A.-M. Vandamme et al. “Managing resistance to Anti-HIV drugs”, Drugs 57:337-361 (1999); R. Schinazi, et al. “Mutations in retroviral genes associated with drug resistance”, Int. Antiviral News 5:129-142 (1997).

Current therapies for HIV infections are typically built around highly active antiretroviral therapy (HAART). HAART therapies are often combinations or “cocktails” of two or more antiretroviral agents. R. M. Gulick, “Current antiretroviral therapy: an overview”, Qual. Life Res. 6:471-474 (1997); K. Henry et al., “Antiretroviral therapy for HIV infection. Heartening Successes mixed with continuing challenges”, Postgrad. Med. 102:100-107 (1997); C. B. Hicks, “Update on antiretroviral therapy”, Radiol. Clin. North Am. 35:995-1005 (1997); R. H. Goldschmidt, “Antiretroviral drug treatment for HIV/AIDS”, Am. Fam. Physician, 54:574-580 (1996). Drugs used in HAART regimens include the nuceloside analogs AZT, stavudine (d4T), and 3TC; nevirapine (a non-nucleoside reverse transcriptase inhibitor, which may be abbreviated NVP), and protease inhibitors such as RTV, SQV, IDV, and nelfinavir. HAART using these treatments may reduce plasma loads of active HIV virus in HIV-1-positive patients to undetectable amounts (below about 50 copies/ml), apparently without the threat of developing resistant strains of HIV. M. Balter, “HIV Survives Drug Onslaught by Hiding Out in T Cells,” Science 278:1227 (Nov. 14, 1997). This document and all documents cited to herein, are incorporated by reference as if fully reproduced below.

The hope was that if active HIV replication was suppressed through HAART for a sufficiently long period, say three years or so, the virus would be completely removed. However, it appears that reducing the plasma concentration of active HIV is not sufficient to eradicate HIV infection completely.

In one aspect, development of drug resistance in HIV-infected patient can be attributed to amplification of existing HIV mutant genotypes, as well as to generation of virions of entirely new genotypes. New anti-HIV drugs with novel chemical moiety and biological activities against these mutants need to be developed in order to control the onset and/or progression of AIDS.

In another aspect, although treatment of HIV-infected patients with potent antiretroviral combination therapy results in a strong decline of the viral loads in peripheral blood, a question—whether this effect is reached in all tissues and different infected cell types is being studied extensively. There are indications that several potential virus reservoirs exist in the body, including lymphoid tissue, central nerve tissue, cerebrospinal fluid, etc. For example, it has been demonstrated that there are reservoirs of integrated and unintegrated HIV existing in memory CD4+ cells.

In three studies, memory CD4⁺ cells were isolated from patients undergoing HAART, most of whom had undetectable plasma HIV-1. Memory CD4⁺ T cells are CD4⁺CD8⁻ T lymphocytes that are “resting” or quiescent. These memory cells are generally non-proliferating, and are capable of being activated in case of a subsequent exposure to an antigen. In this way, they form part of the acquired immune response. Further information describing memory T cells can be found in a standard immunology textbook, such as E. Benjamin, et al., “Immunology: A Short Course,” (1996) (Wiley-Liss). Previous investigators had detected integrated viral DNA in memory T cells, but believed it to be defective. The investigators in the three studies found that once the memory T cells were activated, replication-competent HIV-1 was produced in most cases.

In the first study, replication competent virus was routinely recovered from memory CD4⁺ T lymphocytes of 22 patients who had been treated successfully with HAART for up to 30 months. The frequency of latently infected cells was low, but these frequencies did not decrease with increasing time on therapy, indicating long-term survival of latently infected cells. D. Finzi, et al., “Identification of a Reservoir for HIV-1 in Patients on Highly Active Antiretroviral Therapy”, Science 278:1295 (Nov. 14, 1997).

In the second study, investigators found that highly purified memory CD4+ T cells from patients receiving HAART for an average of ten months were capable of producing infectious virus upon cellular activation in vitro. They also found unintegrated HIV-1 DNA in the memory T cells, which they suggest shows persistent active virus replication in vivo. T-W Chun et al., “Presence of an Inducible HIV-1 Latent Reservoir During Highly Active Antiretroviral Therapy”, Proc. Natl. Acad. Sci. 94:13193-97 (1997).

In the third study, researchers took blood cells from HIV-positive patients undergoing HAART for up to two years and cultured them together with blood cells from HIV-negative donors, along with reagents that trigger memory T cells to become immunologically activated. The researchers observed virus from latently infected memory cells quickly infecting and replicating in the HIV-negative cells, even though the original level of infection of the HIV-positive cells was very low. J. Wong et al., “Recovery of Replication-Competent HIV Despite Prolonged Suppression of Plasma Viremia”, Science 278:1291 (1997).

These results imply that the reservoirs of integrated and unintegrated HIV existing in memory T cells can potentially reestablish active HIV infection and AIDS. These results agree with earlier findings that removing patients from HAART may reestablish active HIV infection and AIDS.

However, conventional HAART does not reach these memory T cells. The drugs that make up HAART's are focused on actively replicating HIV in proliferating T cells and other proliferating immune system cells, such as macrophages. Accordingly, it does not seem likely that continued administration of HAART will reach the cells that are latent reservoirs of HIV infection to eradicate the integrated and unintegrated virus contained within the cells.

There is therefore a need for methods, kits, and compositions that can address the existence of the HIV reservoir in both acutely and chronically infected cells in various tissues of the body.

### SUMMARY OF THE INVENTION

The present invention relates to novel compositions, kits, and methods for treating patients infected with HIV using a combination therapy including 20(S)-camptothecin, an analog of 20(S)-camptothecin, a derivative of 20(S)-camptothecin, a predrug of 20(S)-camptothecin or pharmaceutically active metabolites thereof, collectively referred to herein as CPT and at least one antiretroviral drug, such as nucleoside reverse transcriptase inhibitors, non-nucleoside reverse transcriptase inhibitors, protease inhibitors, fusion inhibitors and integrase inhibitors.

The pharmaceutical composition may include any combinations of CPT with antiretroviral drugs. For example, the pharmaceutical composition may include 1) two nucleoside reverse transcriptase inhibitors and one protease inhibitor; 2) one nucleoside reverse transcriptase inhibitor, one non-nucleoside reverse transcriptase inhibitor, and one protease inhibitor; or 3) one nucleoside reverse transcriptase inhibitor and two protease inhibitors.

The pharmaceutical composition may optionally further include one or more general antiviral agent. Examples of general antiviral agents include, but are not limited to acyclovir, ganciclovir, trisodium phosphonoformate, NOVAPREN, PEPTIDE T OCTAPEPTIDE SEQUENCE, ansamycin LM 427, dextran sulfate, VIRAZOLE, RIBAVIRIN, α-interferon, and β-interferon.

The pharmaceutical composition may also optionally further include one or more immuno-modulator. Examples of immuno-modulator include, but are not limited to immuno-modulator AS-101, BROPIRIMINE, ACEMANNAN, CL246728, EL10, γ-interferon, granulocyte macrophage colony stimulating factor, interleukin-2, α-2-interferon, α-2a-interferon, IMREG-1, IMREG-2, methionine-enkephalin, muramyl-tripeptide granulocyte macrophage colony stimulating factor, rCD4, SK&F106528, and tumor necrosis factor.

The pharmaceutical composition may also optionally further include one or more anti-infection agent. Examples of immuno-modulator include, but are not limited to FLUCONAZOLE, PASTILLE, ORNIDYL, EFLORNITHINE, PIRITREXIM, PENTAMIDINE, ISETHIONATE, spiramycin, and R51211.

A method is provided for treating an HIV-infected host comprising: administering to the HIV-infected host therapeutically effective amount of a composition comprising a compound selected from the group consisting of 20(S)-camptothecin, analog of 20(S)-camptothecin, derivative of 20(S)-camptothecin, predrug of 20(S)-camptothecin and pharmaceutically active metabolite of 20(S)-camptothecin in combination with an effective amount of one or more agents selected from the group consisting of nucleoside reverse transcriptase inhibitor, non-nucleoside reverse transcriptase inhibitor, protease inhibitor, fusion inhibitor and integrase inhibitor.

The routes of administration include, but are not limited to administering or coadministering parenterally, intraperitoneally, intravenously, intraartierally, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

A method is provided for treating an HIV-infected host comprising administering highly active antiretroviral therapy (HAART); and coadministering to the HIV-infected host therapeutically effective amount of a composition comprising a compound selected from the group consisting of 20(S)-camptothecin, analog of 20(S)-camptothecin, derivative of 20(S)-camptothecin, predrug of 20(S)-camptothecin and pharmaceutically active metabolite of 20(S)-camptothecin.

The HAART regimen may be a wide variety of combinations or cocktails of antiretroviral drugs, such as nucleoside reverse transcriptase inhibitors, non-nucleoside HIV reverse transcriptase inhibitors, protease inhibitors, fusion inhibitors and integrase inhibitors. For example, HAART cocktails may include 1) two nucleoside reverse transcriptase inhibitors and one protease inhibitor; 2) one nucleoside reverse transcriptase inhibitor, one non-nucleoside reverse transcriptase inhibitor, and one protease inhibitor; or 3) one nucleoside reverse transcriptase inhibitor and two protease inhibitors.

A method is also provided for ex vivo or in vitro treatment of blood derived cells, bone marrow transplants, or other organ transplants comprising: treating the blood derived cells, bone marrow transplants, or other organ transplants by a pharmaceutical composition comprising: a compound selected from the group consisting of 20(S)-camptothecin, analog of 20(S)-camptothecin, derivative of 20(S)-camptothecin, predrug of 20(S)-camptothecin and pharmaceutically active metabolite of 20(S)-camptothecin in combination with one or more agents selected from the group consisting of nucleoside reverse transcriptase inhibitor, non-nucleoside reverse transcriptase inhibitor, protease inhibitor, fusion inhibitor and integrase inhibitor.

A kit is provided for the treatment of HIV-infected host comprising: a composition comprising a compound selected from the group consisting of 20(S)-camptothecin, analog of 20(S)-camptothecin, derivative of 20(S)-camptothecin, prodrug of 20(S)-camptothecin and pharmaceutically active metabolite of 20(S)-camptothecin; and a cocktail of two or more agents selected from the group consisting of nucleoside reverse transcriptase inhibitors, non-nucleoside reverse transcriptase inhibitors, protease inhibitors, fusion inhibitors and integrase inhibitors.

According to any one of the above pharmaceutical compositions, kits and methods, CPT may be 20(S)-camptothecin or any analog or derivative of 20(S)-camptothecin. Examples of 20(S)-camptothecin analogs include, but are not limited to 9-nitro-20(S)-camptothecin and 9-amino-20(S)-camptothecin. Examples of 20(S)-camptothecin derivatives include, but are not limited to 9-methyl-camptothecin, 9-chloro-camptothecin, 9-flouro-camptothecin, 7-ethyl camptothecin, 10-methyl-camptothecin, 10-chloro- camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, 10,11-ethylenedioxy camptothecin, 7-(4-methylpiperazinomethylene)-10,11-methylenedioxy camptothecin, camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate.

According to any one of the above pharmaceutical compositions, kits and methods, the at least one nucleoside reverse transcriptase inhibitor, non-nucleoside reverse transcriptase inhibitor, or protease inhibitor may be any of these antiretroviral drugs or combination thereof. Examples of nucleoside reverse transcriptase inhibitors include, but are not limited to ZIDOVUDINE, DIDANOSINE, ZALCITABINE, LAMIVUDINE, STAVUDINE, ABACAVIR, and ADEFOVIR DIPIVOXIL. Examples of non-nucleoside reverse transcriptase inhibitors include, but are not limited to NEVIRAPINE, DELAVIRDINE, and EFAVIRENZ. Examples of protease inhibitors include, but are not limited to, INDINAVIR, RITONAVIR, SAQINAVIR, NELFINAVIR, and AMPRENAVIR. Examples of fusion inhibitors include, but are not limited to DP107, DP178 and T-20. Examples of integrase inhibitors include, but are not limited to L-731, 988, L-708,906, L-731,927, and L-731,942.

### DETAILED DESCRIPTION OF THE INVENTION

The present invention provides new and improved pharmaceutical compositions, kits and methods for treating HIV infection using a combination therapy which includes 20(S)-camptothecin, an analog of 20(S)-camptothecin, a derivative of 20(S)-camptothecin, a predrug of 20(S)-camptothecin or pharmaceutically active metabolites thereof collectively referred to herein as CPT. At least one nucleoside HIV reverse transcriptase inhibitor, non-nucleoside HIV reverse transcriptase inhibitor, HIV protease inhibitor, or combinations thereof is combined with CPT to achieve therapeutic synergistic effects in the treatment of HIV-infected patients.

1. Camptothecin, Analogs and Derivatives

Camptothecin was isolated from the plant, Camptotheca acuminata, in the 1960's (Wall, M. et al. (1966) J. Am. Chem. Soc. 88: 3888-3890). Camptothecin has a pentacyclic ring system with only one asymmetric center in ring E with a 20(S)-configuration. The pentacyclic ring system includes a pyrrole quinoline moiety (rings A, B and C), a conjugated pyridone (ring D), and a six-membered lactone (ring E) with an α-hydoxyl group. Continuous clinical trials of camptothecin, its analogs and derivatives have been undertaken for over two decades, which demonstrated clinical anti-cancer efficacy of CPT in the treatment various types of tumors and malignancies.

Camptothecin and its derivatives have been shown to inhibit DNA topoisomerase I by stabilizing the covalent complex (“cleavable complex”) of enzyme and strand-cleaved DNA. Inhibition of topoisomerase I by camptothecin induces protein-associated DNA single-strand breaks which occur during the S-phase of the cell cycle. Since the S-phase is relatively short compared to other phases of the cell cycle, longer exposure to camptothecin should result in increased cytotoxicity of tumor cells.

Preliminary in vitro studies of inhibitory effects of camptothecin on HIV infection of human H9 cells demonstrated that camptothecin significantly reduced the activity of the viral-associated topoisomerase I, not that of topoisomerase II. E. Priel, et al. “Inhibition of human immunodeficiency virus (HIV-1) replication in vitro by noncytotoxic doses of camptothecin, a topisomerase I inhibitor”, AIDS Res. Hum. Retroviruses 7:65-72 (1991). It has also been found that camptothecin inhibits equine infectious anemia virus (EIAA) replication in chronically infected cells and Moloney murine leukemia virus (MMLV) replication, suggesting that camptothecin may act at a common, but as yet identified, step in the life cycle of retrovirus. Based on several findings showing the involvement of topoisomerase I in the recombinational process, it was hypothesized that topoisomerase might participate in retroviral integration.

The inhibitory effects of CPT on HIV infection were also performed in cell lines that were latently infected with HIV-1 or transfected with HIV-1 LTR-reporter gene expression vectors. For example, 20(S)-camptothecin has been shown to have inhibitory effects on the expression of downstream reporter gene under the control of HIV-1 LTR in human CD4+ lymphocytic cell line RPMI 8402. C. J. Li “Camptothecin inhibits Tat-mediated transactivation of type 1 human immunodeficiency virus” J. Biol. Chem. 269:7051-7054 (1994). Interestingly, the effect of camptothecin on HIV-1 LTR in these cell lines was due to selective inhibition rather than cytotoxic effects, since cell survival was not significantly affected by the drug at the concentration used. In addition, camptothecin did not inhibit the promoter activity of rous sarcoma virus (RSV) or expression of gro, a cellular gene. It has been speculated that DNA topoisomerase I influences Tat/TAR-mediated transcription by selectively interacting with Tat/TAR or their associated proteins. Another speculation was that the inhibition by camptothecin of HIV-1 LTR is independent of its inhibition of DNA topoisomerase I; the target may be a novel cellular factor, probably a Tat- or TAR-associated protein.

Topotecan, a semisynthetic analog of camptothecin, was shown to inhibit both acute and chronic HIV-1 infections in vitro. J. L. Zhang, et al. “Topoisomerase inhibits human immunodeficiency virus type 1 infection through a topisomerase-independent mechanism in a cell line with altered topoisomerase I” Antimicrob. Agents Chemother. 41:977-981 (1997). The antiviral effects of topotecan were observed not only in the topoisomerase-mutated CPT-K5 cell line but also in peripheral blood mononuclear cells (PBMC) acutely infected with clinical isolates and in OM10.1 cells latently infected with HIV and activated by tumor necrosis factor alpha (TNF-α). It was again hypothesized that this camptothecin targets factors in virus replication other than cellular topoisomerase I and inhibits cytokine-mediated activation in latently infected cells by means other than cytotoxicity.

“Camptothecin”, as it is referred to in the present invention, includes the plant alkaloid 20(S)-camptothecin, water insoluble or soluble analogs and derivatives of 20(S)-camptothecin, prodrugs of camptothecin, and metabolites of 20(S)-camptothecin. Examples of camptothecin derivatives include, but are not limited to, 9-nitro-20(S)-camptothecin, 9-amino-20(S)-camptothecin, 9-methyl-camptothecin, 9-chloro-camptothecin, 9-flouro-camptothecin, 7-ethyl camptothecin, 10-methyl-camptothecin, 10-chloro--camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin. Prodrugs of camptothecin include, but are not limited to, esterified camptothecin derivatives as decribed in U.S. Pat. No. 5,731,316, such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate.

Native, unsubstituted, camptothecin can be obtained by purification of the natural extract, or may be obtained from the Stehlin Foundation for Cancer Research (Houston, Tex.). Substituted camptothecins can be obtained using methods known in the literature, or can be obtained from commercial suppliers. For example, 9-nitro-camptothecin may be obtained from SuperGen, Inc. (San Ramon, Calif.), and 9-amino-camptothecin may be obtained from Idec Pharmaceuticals (San Diego, Calif.). Camptothecin and various of its analogs and derivatives may also be obtained from standard fine chemical supply houses, such as Sigma Chemicals.

2. Pharmaceutical Compositions of the Present Invention

According to the present invention, novel pharmaceutical compositions are provided for the treatment of HIV-infected patients in the clinic. In essence, the pharmaceutical compositions are combination of CPT and at least one nucleoside HIV reverse transcriptase inhibitor, non-nucleoside HIV reverse transcriptase inhibitor, HIV protease inhibitor, and combination thereof. These HIV inhibitors are preferably drugs clinically proven to have anti-HIV efficacy, more preferably those drugs used in the “cocktail” treatment of HIV-infected and/or AIDS patients.

The inventors believe that the combination of CPT with anti-HIV drugs should have therapeutic synergism in the treatment of HIV infection, i.e. asserting superior therapeutic effects on viruses and HIV-infected cells than any one of the drugs administered alone or the current cocktails of the anti-retroviral drugs used in the clinic (or in HAART).

A combination therapy including CPT and anti-retroviral drugs represents a new approach for treating HIV-infected and/or AIDS patients, presumably due to the different mechanisms of action of CPT and/or toxicity profiles. Most anti-retroviral drugs used in the clinic interfere with the functions of different enzymatic components of HIV, such as reverse transcriptase and protease. CPT may assert its therapeutic effects predominantly at the cellular level through topoisomerase-dependent or -independent mechanisms of actions. CPT may also inhibit strand transfer, one of the catalytic functions of HIV integrase. For example, CPT may bind to the specific conformation adopted by HIV integrase in the formation of a stable, active strand transfer complex, thus preventing integration of HIV viral DNA into the host genome. Further, unlike anti-AIDS drugs currently used in the clinic, CPT has been shown to be highly active against HIV in both acutely and chronically infected cells. Moreover, CPT has also been shown to be active against HIV mutants resistant to the nucleoside reverse transcriptase inhibitor, AZT, as well as against HIV strains sensitive to AZT.

The inventors believe that CPT should work in concert with anti-retroviral drugs to inhibit HIV infection, reduce viral loads, and eradicate HIV-infected cells in the body. With its well-established functions against cancer cells and preliminarily demonstrated activity against HIV-infected cells, CPT should have activity against the cells that are the latent reserviors of HIV infection, ultimately resulting in elimination of replication competent HIV and preventing relapse of HIV infection. Furthermore, the anti-cancer activity of CPT may confer a dual therapeutic advantage both in suppression of HIV replication and eradication of cancer cells in AIDS-associated malignanies such as Kaposi's sarcoma, and Hodgkins and non-Hodgkins lymphoma.

The pharmaceutical compositions of the present invention are believed to be useful in the prevention or treatment of infection by HIV and the treatment of, and delaying of the onset of consequent pathological conditions such as AIDS. Treating AIDS or preventing or treating infection include, but is not limited to, treating a wide range of states of HIV infection: AIDS, ARC (AIDS related complex), both symptomatic and asymptomatic, and actual or potential exposure to HIV. For example, the pharmaceutical composition of the present invention are believed to be useful in treating infection by HIV after suspected past exposure to HIV, e.g., blood transfusion, organ transplant, exchange of body fluids, bites, accidental needle stick, or exposure to patient blood during surgery.

It should be noted that the pharmaceutical compositions of the present invention are not limited to the treatment of HIV infection and/or AIDS. These compositions may also be used for the treatment of other viral infections, such as EIAV, MoMuLV, human retroviruses HTLV-I/II which have been implicated in adult T cell leukemia/lymphoma and neurological diseases, tropical spastic paraparesis or HTLV-I associated myelopathy, hepatitis viruses, etc.

A wide variety of anti-retroviral drugs can be used in combination with CPT. Many small molecule (e.g. organic compounds) and macromolecule (antisense DNAs/RNAs, ribozymes, viral surface protein-binding proteins or nucleotides, etc.) Drugs against HIV have been developed since the discovery of correlation between HIV and AIDS. In particular, many drugs have been developed to target critical enzymes of retroviruses and inhibit replication of the virus inside the host cell. For example, nucleoside or nucleotide analogs such as AZT, dideoxycytidine (ddC), and dideoxyinosine (ddI) were developed to inhibit reverse transcriptase (RT) of retroviruses by acting as competitive inhibitors and chain terminators. Non-nucleoside or nucleotide inhibitors have also been found to inhibit reverse transcriptase activity of retroviruses by exerting an allosteric effect by binding to a hydrophobic pocket close to the active site of RT. The protease (PRO) inhibitors in current use are targeted at the active site of the enzyme.

In addition to the RT and PRO inhibitors of HIV infection, other classes of antiviral agents targeting different components of HIV or interfering with different stages of HIV life cycle may be also be used in conjunction with CPT to achieve efficacious clinical results. For example, synthetic peptides have been modeled to mimic the coiled-coiled helical bundle formed by heptad repeat sequences of one of the two subunits of HIV envelop glycoprotein, the transmembrane glycoprotein (gp41). Wild C. T. et al. “A synthetic peptide inhibitor of HIV replication: correlation between solution structure and viral inhibition” Proc. Natl. Acad. Sci. USA 89: 10537-10541 (1992). These heptad sequences play important roles in the conformational changes essential for membrane fusion of HIV with host cells. The synthetic peptides, DP107 and DP178, have been shown to inhibit infection in vitro by disrupting the gp41 conformational changes associated with membrane fusion. Wild, C. et al. “Peptides corresponding to a predictive alpha-helical domain of HIV-1 gp41 are potent inhibitors of virus infection” Proc. Natl. Acad. Sci. USA 91: 9770-9774 (1994). In particular, a 36-amino acid peptide (T-20), corresponding to DP178, functions as a potent inhibitor of the HIV-1 envelop-cell membrane fusion and viral entry. Wild, C. et al. “A synthetic peptide from HIV-1 gp41 is a potent inhibitor of virus-mediated cell-cell fusion” AIDS Res. Hum. Retroviruses 9:1051-1053 (1993). When used in monotherapy, T-20 demonstrated potent antiviral activity in vivo when administered as an intravenous subcutaneous infusion in trials of 28 days or less. Lalezari, J. et al “Safety, pharmacokinetics, and antiviral activity of T-20 as a single agent in heavily pretreated patients” 6^{th} Conference on Retroviruses and Opportunistic Infections, Chicago, February 1999 [Abstract LB13]. Such inhibitors of HIV fusion and entry into the host cells may be combined with CPT, as well as other anti-retroviral agents to inhibit HIV infection at different stages of the retroviral life cycle.

Further, inhibitors of retroviral integrase may be used in conjunction with CPT according to the present invention. A variety of inhibitors of HIV integrase have been identified that inhibit HIV integration at different stages. In general, retroviral integration occurs in the following three biochemical stages: 1) assembly of a stable complex with specific DNA sequences at the end of the HIV-1 long terminal repeat (LTR) regions, (2) endonucleolytic processing of the viral DNA to remove the terminal dinucleotide from each 3′ end, and (3) strand transfer in which the viral DNA 3′ ends are covalently linked to the cellular (target) DNA. Pommier, Y. and Neamati, N. in Advances in Viral Research, K. Maramorosch, et al. eds. Academic Press, New York (1999), pp 427-458. Compounds have been identified to interfere with assembly of the stable complex in assays with purified, recombinant integrase. Hazuda, D. J. et al. Drug Des. Discovery 15: 17 (1997). In a random screening of more than 250,000 samples.

A variety of compounds have been discovered as inhibitors of strand transfer reaction catalyzed by integrase. Hazuda, D. J. et al. “Inhibitors of strand transfer that prevent integration and inhibit HIV-1 replication in cells” Science 287:646-650 (2000). The most potent and specific compounds each contained a distinct diketo acid moiety, such as compound L-731,988, L-708,906, L-731,927, and L-731,942. Hazuda, D. J. et al. (2000), supra. Such inhibitors of HIV integration into the host genome may be combined with CPT, as well as other anti-retroviral agents to inhibit HIV infection at different stages of the retroviral life cycle.

In the pharmaceutical compositions of the present invention, nucleoside reverse transcriptase inhibitors, non-nucleoside reverse transcriptase inhibitors, protease inhibitors, fusion inhibitors and integrase inhibitors are the preferred anti-retroviral drugs in combination with CPT. Examples of the nucleoside HIV reverse transcriptase inhibitor include, but are not limted to ZIDOVUDINE (AZT), DIDANOSINE (ddI), ZALCITABINE (ddC), LAMIVUDINE (3TC), STAVUDINE (d4T), ABACAVIR (1592U89), and ADEFOVIR DIPIVOXIL (bis(POM)-PMEA). Examples of the non-nucleoside HIV reverse transcriptase inhibitor include, but are not limited to NEVIRAPINE (BI-RG-587), DELAVIRDINE (BHAP, U-90152) and EFAVIRENZ (DMP 266). Examples of the HIV protease inhibitors include, but are not limited to INDINAVIR (MK-639), RITONAVIR (ABT-538), SAQINAVIR (Ro-31-8959), NELFINAVIR (AG-1343), and AMPRENAVIR (141W94).

The pharmaceutical compositions of the present invention include CPT in combination with any one or more of the antiretroviral drugs, preferably with a “cocktail” of nucleoside reverse transcriptase inhibitors, non-nucleoside HIV reverse transcriptase inhibitors, and protease inhibitors. For example, CPT may be combined with two nucleoside reverse transcriptase inhibitors (e.g. ZIDOVUDINE (AZT) and LAMIVUDINE (3TC)), and one protease inhibitor (e.g. INDINAVIR (MK-639)). CPT may also be combined with one nucleoside reverse transcriptase inhibitor (e.g. STAVUDINE (d4T)), one non-nucleoside reverse transcriptase inhibitor (e.g. NEVIRAPINE (BI-RG-587)), and one protease inhibitor (e.g. NELFINAVIR (AG-1343)). Alternatively, CPT may be combined with one nucleoside reverse transcriptase inhibitor (e.g. ZIDOVUDINE (AZT)), and two protease inhibitors (e.g. NELFINAVIR (AG-1343) and SAQINAVIR (Ro-31-8959)).

Optionally, the pharmaceutical composition of the present invention further includes one or more general antiviral agents. Examples of general antiviral agents include, but are not limited to acyclovir, ganciclovir, trisodium phosphonoformate, NOVAPREN (Novaferon Labs, Inc., Akron, Ohio), PEPTIDE T OCTAPEPTIDE SEQUENCE (Peninsula Labs, Belmont, Calif.), ansamycin LM 427 (Adria Labortories, Dublin, Ohio), dextran sulfate, VIRAZOLE, RIBAVIRIN ((Virateck/ICN, Costa Mesa, Calif.), α-interferon, and β-interferon. General antiviral agents can be used to prevent or inhibit opportunistic infections of other viruses.

Also optionally, the pharmaceutical composition of the present invention may further include one or more immuno-modulator. Examples of the immuno-modulator include, but are not limited to AS-101 (Wyeth-Ayerst Labs, Philadelphia, Pa.), BROPIRIMINE (Upjohn, Kalamazoo, Mich.), ACEMANNAN (Carrington Labs, Inc., Irvine, Tex.), CL246728 (American Cyanamid, Pearl River, N.Y.), EL10 (Elan Corp, Gainesville, Ga.), γ-interferon, granulocyte macrophage colony stimulating factor, interleukin-2, α-2-interferon, α-2a-interferon, IMREG-1, IMREG-2 (Imreg, New Orleans, La.), methionine-enkephalin, muramyl-tripeptide granulocyte macrophage colony stimulating factor, rCD4, SK&F106528 (Smith, Kline & French Laboratories, Philadelphia, Pa.), and tumor necrosis factor. The immuno-modulator can be used to stimulate and activate replication of latent HIV which eventually leads to apoptosis of the infected cells.

Also optionally, the pharmaceutical composition of the present invention may further include one or more general anti-infection agent. Examples of the general anti-infection agent include, but are not limited to FLUCONAZOLE (Pfizer, New York, N.Y.), PASTILLE (Squibb Corp, Princeton, N.J.), ORNIDYL, eflornithine (Merrell Dow, Cincinnati, Ohio), PIRITREXIM (Burroughs Wellcome, Research Triangle Park, N.C.), pentamidine (Fisons Corporation, Bedford, Mass.), isethionate, spiramycin (Rhone-Poulenc Pharmaceuticals, Princeton, N.J.), and Intraconazole-R51211 (Janssen Pharmaceuticals, Piscataway, N.J.). General anti-infection agents can be used to treat opportunistic infections of bacteria, parasites and other organisms in HIV-infected patients.

3. Formulation and Administration of Pharmaceutical Compositions of the Present Invention

According to the present invention, the pharmaceutical compositions are combinations of CPT and antiretroviral drug(s). Formulation of the composition for clinical use will vary according to the particular type of CPT and antiretroviral drug(s). Dosage amounts and frequency will also vary according to the formulation, and individual patient characteristics. Generally, determining dosage forms, dosage amount and frequency can be accomplished using conventional pharmacological formulations, clinical dosing studies, coupled with appropriate diagnostics.

For injection, the pharmaceutical compositions can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. If formulated in aqueous solutions, physiologically compatible buffers such as Hanks' solution, Ringer's solution, or physiological saline buffer are preferred. For transmucosal administration, penetrants appropriate to the barrier to be permeated are used in the formulation. Such penetrants are generally known in the art.

For oral administration, the pharmaceutical compositions can be formulated readily by combining with pharmaceutically acceptable carriers that are well known in the art. Such carriers enable the compounds to be formulated as tablets, pills, dragees, capsules, emulsions, lipophilic and hydrophilic suspensions. liquids, gels, syrups, slurries, suspensions and the like, for oral ingestion by a patient infected with HIV. Pharmaceutical preparations for oral use can be obtained by mixing the composition of the present invention with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylprrolidone (PVP). If desired, disintegrating agents may be added, such as the cross-linked polyvinyl pyrrolidone, agar, or alginic acid or a salt thereof such as sodium alginate.

In the oral dosage forms, it may be useful to include antioxidants or preservatives. Antioxidants that may be used are sodium sodium sulphite, sodium hydrogen sulphite, sodium metabisulphite, ascorbic acid, ascorbylpalmitate, -myristate, -stearate, gallic acid, gallic acid alkyl ester, butylhydroxyamisol, nordihydroguaiaretic acid, tocopherols as well as synergists (substances which bind heavy metals through complex formation, for example lecithin, ascorbic acid, phosphoric acid ethylene diamine tetracetic acid, citrates, tartrates). Addition of synergists substantially increases the antioxygenic effect of the antioxidants.

Preservatives may also be used in the oral dosage forms. Examples of preservatives include sorbic acid, p-hydroxybenzoic acid esters (for example lower alkyl esters), benzoic acid, sodium benzoate, trichloroisobutyl alcohol, phenol, cresol, benzethonium chloride, chlorhexidine and formalin derivatives.

Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active drug doses.

Pharmaceutically preparations of the pharmaceutical compositions of the present invention which can be used orally include push-fit capsules made of gelatin, as well as soft, sealed capsules made of gelatin and a plasticizer, such as glycerol or sorbitol. The push-fit capsules can contain the adenosine analog in admixture with filler such as lactose, binders such as starches, and/or lubricants such as talc or magnesium sterate and, optionally, stabilizers. In soft capsules, the adenosine analog may be dissolved or suspended in suitable liquids, such as fatty oils, liquid paraffin, or liquid polyethylene glycols. In addition, stabilizers may be added. All formulations for oral administration should be in dosage suitable for such administration.

For buccal administration, the pharmaceutical compositions may take the form of tablets or lozenges formulation in conventional manner.

For administration by inhalation, pharmaceutical compositions of the present invention may be conveniently delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providng a valve to deliver a metered amount. Capsules and cartridges of, for example, galetin for use in an inhaler or insulator may be formulated containing a powder mix of the compound and suitable powder base such as lactose or starch.

The pharmaceutical compositions of the present invention may be administrated parenterally, e.g. by bolus injection or continuous infusion. Formulations for injection may be presented in unit dosage form, such as in ampules or in multidose containers, with an added preservative. The formulations may take such forms as suspension, solutions or emulsion in oily or aqueous vehicles, and may contain formulatory agents such as suspending, stabilizing and/or dispersing agents.

Pharmaceutical formulations for the pharmaceutical compositions for parenteral administration include aqueous solutions of the CPT and antiretroviral drug(s) in a water-soluble form. Additionally, suspensions of CPT and antiretroviral drug(s) may be prepared as appropriate oily injection suspension. Suitable lipophilic solvents or vehicles include fatty oils such as sesame oil, or syntheic fatty acid esters, such as ethyl oleate or triglycerides, or liposomes. Aqueous injection may contain substances which increase the viscosity of the suspension, such as sodium carboxymethyl cellulose, sorbitol, or dextran. Optionally, the suspension may also contain suitable solubilizers or agents which increase the solubility of the compounds to allow for the preparation of highly concentrated solutions. Examples of such solubilizers include, but are not limited to, cyclodextrin such as α-, β-, and γ-cyclodextrin and modified, amorphous cyclodextrin such as hydroxy-substituted α, β-, and γ-cyclodextrin. Alternatively, the active ingredient may be in powder form for constitution with a suitable vehicle, e.g., sterile pyrogen-free water, before use.

The pharmaceutical compositions of the present invention may also be formulated in rectal compositions such as suppositories or retention enemas, e.g. containing conventional suppository bases such as cocoa butter, carbowaxes, polyethylene glycols or other glycerides, all of which melt at body temperature, yet are solidified at room temperature.

In addition, the pharmaceutical compositions of the present invention may be formulated as a depot preparation for administration by implantation (e.g., subcutaneously or intramuscularly) or by intramuscular injection. Thus, for example, the pharmaceutical compositions may be formulated with suitable polymeric or hydrophobic materials (e.g., as an emulsion in an acceptable oil) or ion exchange resins, or as sparingly soluble derivative, for example, as sparingly soluble salt.

Alternatively, the pharmaceutical compositions of the present invention may be administrated to a HIV-infected patient by employing other delivering systems such as liposome-mediated drug delivery. Liposomes and emulsions are well known examples of delivery vehicles or carriers for hydrophobic drugs. Certain organic solvents such as dimethylsulfoxide also may be employed, although usually at the cost of greater toxicity. Additionally, the adenosine analogs can be delivered using a sustained-release system, such as semipermeable matrices of solid hydrophobic polymers containing the adenosine analog. Various types of sustained-release materials have been established and are well known by those skilled in the art. Sustained-release capsules may, depending on their chemical nature, release the adenosine analog for a few weeks up to over 100 days.

In addition, the pharmaceutical compositions of the present invention may be administrated in a targeted drug delivery system, for example, in a liposome coated with a cell-specific antibody. Such liposomes will be targeted to and taken up selectively by the cell of interest (a specific subset of T cells). Liposomes and emulsions are well known examples of delivery vehicles or carriers for hydrophobic drugs. For example, long-circulating, i.e., stealth, liposomes can be employed. Such liposomes are generally described in U.S. Pat. No. 5,013,556, the teaching of which are hereby incorporated by reference.

Alternatively, the pharmaceutical compositions of the present invention may also be administrated with various agents to reduce acid concentration in the stomach. This reduces acid lability and allows for enhanced concentrations of the pharmaceutical composition for enhanced gastric and/or intestinal absorption.

4. Methods of Treatinq HIV Infection with Combination Therapy Including CPT

The present invention also provides methods for treating HIV infection with combination therapy by administering the pharmaceutical compositions described above, or by coadministering CPT and antiretroviral drugs in separate dosage forms.

In one embodiment, the method for treating HIV-infected host comprises: administering to the HIV-infected host therapeutically effective amount of a pharmaceutical composition comprising a compound selected from the group consisting of 20(S)-camptothecin, analog of 20(S)-camptothecin, derivative of 20(S)-camptothecin, prodrug of 20(S)-camptothecin and pharmaceutically active metabolite of 20(S)-camptothecin, and at least one of the agent selected from the group consisting of nucleoside reverse transcriptase inhibitors, non-nucleoside reverse transcriptase inhibitors, protease inhibitors, fusion inhibitors and integrase inhibitors. The dosage forms and routes of administration are described in Section 3.

In another embodiment, the method for treating HIV-infected host comprises: administering to the HIV-infected host therapeutically effective amount of a composition comprising a compound selected from the group consisting of 20(S)-camptothecin, analog of 20(S)-camptothecin, derivative of 20(S)-camptothecin, prodrug of 20(S)-camptothecin and pharmaceutically active metabolite of 20(S)-camptothecin in combination with an effective amount of one or more agents selected from the group consisting of nucleoside reverse transcriptase inhibitors, non-nucleoside reverse transcriptase inhibitors, protease inhibitors, fusion inhibitors and integrase inhibitors.

The routes of administration includes, but are not limited to, administering or coadministering parenterally, intraperitoneally, intravenously, intraartierally, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

For example, oral administration may be a preferred route of administration for camptothecin analogs 9-nitro-20(S)-camptothecin, and 9-amino-20(S)-camptothecin. More preferably, oral dosage forms of CPT are coadministered with cocktails of antiretroviral drugs including nucleoside reverse transcriptase inhibitors, non-nucleoside HIV reverse transcriptase inhibitors, protease inhibitors, fusion inhibitors and/or integrase inhibitors.

In yet another embodiment of the present invention, a method of treating an HIV-infected host comprises: administering highly active antiretroviral therapy (HAART); and coadministering to the HIV-infected host therapeutically effective amount of a composition comprising a compound selected from the group consisting of 20(S)-camptothecin, analog of 20(S)-camptothecin, derivative of 20(S)-camptothecin, prodrug of 20(S)-camptothecin and pharmaceutically active metabolite of 20(S)-camptothecin.

According to this embodiment, an HIV positive patient receives HAART, together with appropriate pharmaceuticals, such as antivirals; antifungals; and antibiotics, to protect against opportunistic infections. Additionally, the patient is coadministered CPT, according to the invention. This regimen is continued for a period past the point when the levels of integrated and unintegrated HIV in active and memory T cells are undetectably low. At the end of the period, the patient is weaned from HAART and from CPT according to the invention. At this point, the patient is monitored for reestablishment of normal immune function and for signs of reemergence of HIV infection. Additionally, any needed conjunctive immunotherapy, such as bone marrow transplants, various cytokines or vaccination, is administered. If there are no signs of HIV infection for a suitable period, then the patient is weaned from the pharmaceuticals that protect against opportunistic infections. After this, the patient is monitored on a routine basis for life to detect reemergence of HIV infection, in which case repeat therapy according to the above preferred embodiment must be undertaken

The various aspects of practicing the invention will now be discussed in more detail. Patients suffering from HIV infections are often treated using a combination of HAART and various other pharmaceuticals. These other pharmaceuticals may be coadministered with the HAART for a variety of reasons, including treating the opportunistic infections that can be common in HIV patients. However, recent findings suggest that even after 30 months of HAART and undetectable viral load, patient-derived lymphocytes that are actively producing virus can be cultured in vitro. D. Finzi, et al. “Identification of a reservoir for HIV-1 in patients on highly active antiretroviral therapy”, Science, 278:1295-300 (1997). The recovered virus did not contain resistance-related mutations, indicating that virus replication had indeed been greatly suppressed. To survive HIV infection, patients will require permanent HAART. Long-term treatment might ultimately result in multidrug-resistant virus, leaving few options for the so-called “salvage therapy”.

The present invention also provides a novel treatment regimen for patients infected with HIV. By combining HAART with CPT coadministration, circulating virus in peripheral blood and latent virus hidden in the reservoirs in resting T cells may be eradicated through concerted inhibition of viral replication by HAART and induction of cell death by CPT.

According to this embodiment, CPT may be coadministered with any HAART regimen. The current standard of care using HAART is usually a combination of at least three nucleoside reverse transcriptase inhibitors and frequently includes a protease inhibitors, or alternatively a non-nucleoside reverse transcriptase inhibitor. Patients who have low CD4⁺ cell counts or high plasma RNA levels may require more aggressive HAART. For patients with relatively normal CD4⁺ cell counts and low to non-measurable levels of plasma HIV RNA over prolonged periods (i.e. slow or non-progressors) may require less aggressive HAART. For antiretroviral-naive patients who are treated with initial antiretroviral regimen, different combinations (or cocktails) of antiretroviral drugs can be used.

Preferably, CPT may be coadministered with a “cocktail” of nucleoside reverse transcriptase inhibitors, non-nucleoside HIV reverse transcriptase inhibitors, and protease inhibitors. For example, CPT may be coadministered with a cocktail of two nucleoside reverse transcriptase inhibitors (e.g. ZIDOVUDINE (AZT) and LAMIVUDINE (3TC)), and one protease inhibitor (e.g. INDINAVIR (MK-639)). CPT may also be coadministered with a cocktail of one nucleoside reverse transcriptase inhibitor (e.g. STAVUDINE (d4T)), one non-nucleoside reverse transcriptase inhibitor (e.g. NEVIRAPINE (BI-RG-587)), and one protease inhibitor (e.g. NELFINAVIR (AG-1343)). Alternatively, CPT may be coadministered with a cocktail of one nucleoside reverse transcriptase inhibitor (e.g. ZIDOVUDINE (AZT)), and two protease inhibitors (e.g. NELFINAVIR (AG-1343) and SAQINAVIR (Ro-31-8959)).

Coadministration in the context of this invention is defined to mean the administration of more than one therapeutic in the course of a coordinated treatment to achieve an improved clinical outcome. Such coadministration may also be coextensive, that is, occurring during overlapping periods of time. Further discussion of such conventional treatment can be found in R. M. Gulick, “Current antiretroviral therapy: an overview”, Qual. Life Res. 6:471-474 (1997); K. Henry et al., “Antiretroviral therapy for HIV infection. Heartening Successes mixed with continuing challenges”, Postgrad. Med. 102:100-107 (1997); C. B. Hicks, “Update on antiretroviral therapy”, Radiol. Clin. North Am. 35:995-1005 (1997); R. H. Goldschmidt, “Antiretroviral drug treatment for HIV/AIDS”, Am. Fam. Physician, 54:574-580 (1996).

The present invention may also serve as an adjunct to this conventional therapy through coadministration. In an alternative embodiment, the methods of present invention may be practiced apart from conventional therapy, if appropriate. The nature of the invention is such that the administration of a pharmaceutical composition combining CPT and antiretrovial drugs or coadministration of CTP along with cocktails of antiviral drugs, should have a superior antiviral effect. Such an antiviral effect may be additive or it may have synergistic effects on the patient. In either situation, performing treatment on HIV-infected patients according to the invention is an important advance because the inventive treatment is focused not only on inhibiting replicating virus but also on eradicating infected cells harboring latent virus, such as memory T cells.

Memory cells are a particularly difficult target to reach with most conventional anti-HIV therapies employing antiretroviral drugs such as reverse transcriptase inhibitors and retroviral protease inhibitors. As noted above, such therapies are most effective against HIV in proliferating cells. Such cells are much more interactive with their environment, and thus offer more opportunity for exogenous intervention. In fact, prior to this invention, approaches to HIV therapy were focused on such proliferating cells almost exclusively, because of the relative ease of intervention.

However, to reach resting/memory T cells, which are by definition non-proliferating until activated, non-conventional approaches are needed. Accordingly, the invention provides for combination therapy that combines cocktail treatment of HIV infection with CPT that has cytotoxic effects with respect to memory cells. These inventive approaches are characterized by their differential ability to affect non-proliferating T lymphocytes, as compared to conventional HIV therapies.

Coadministration of CPT into HIV-infected patients may inhibit and/or eliminate HIV infection by a variety of mechanisms of actions. In the course of further discussing the invention, the inventor does not wish to be bound by a particular mechanism or explanation of action, as such understanding is not necessary for the practice of the invention. Within this context, however, the inventor hypothesizes that, for example, coadministration of CPT may intervene in essential cellular structure that is not involved in cell replication. In one instance, CPT may accelerate non-replicating DNA strand breaks, consequently inducing apoptosis. Additionally, CPT may also induce lysis of resting/memory cells by disrupting the membrane of the memory cells. Moreover, CPT may selectively induce apoptosis in HIV-infected cells and yet remain cytostatic to uninfected lymphocytic cells.

Alternatively, CPT may selectively activate apoptotic genes in memory cells, resulting in programmed cell death. For example, CPT may induce activation of NK-κB, which leads to downstream cascade of signal transduction and eventually apoptosis.

Coadministration of CPT with HAART in the presence of immuno-stimulant TNF may work in concert to eradicate the host's reservoir of memory cells. Such activated T cells may begin proliferating, thus exposing any integrated or unintegrated HIV to conventional HAART. This allows use of HAART to eliminate or reduce the reservoir of HIV contained in the memory cell pool.

There may be different therapeutic responses in the patients depending on the particular CPT used. It is possible that although analogues or derivatives of 20(S)-camptothecin are derived from the same parent compound, these CPTs may differ in several properties including physical characteristics, pharmacokenetics, metabolism, and toxicities. However, in essence, these CPTs assert their inhibitory effects at the cellular level, i.e. capable of eliminating HIV-infected cells harboring latent virus. This is a particular sign of the non-obviousness of this invention that the effect of such therapeutic agents on lymphocytes such as CD4⁺ T cells was previously seen as a deleterious side effect, rather than a desirable property. This is because, for example, the apparent objective of previous anti-HIV therapies was to eradicate the virus without doing further damage to the patient's immune system. In the case of the present invention, a portion of the patient's immune system is actually further damaged in order to reduce or eliminate a previously unreachable reservoir of HIV.

Camptothecin and its analogues have been used in the clinic for a wide varities of tumors and malignancies. In these oncological applications, any potential side effects that differentially targeted the immune system, such as loss of acquired immunity, were seen as undesirable. However, in the context of this invention, loss of acquired immunity through the elimination of latent viral reservoirs in resting T cells, is a potentially desirable condition.

After, or during, administering or coadministering CPT according to the invention, It may be desirable, in certain circumstances, to continue HAART. Additionally, it may be desirable to continue administering or coadministering drugs for treating the opportunistic infections that can be common in HIV patients. Continuing such treatments helps to keep active virus levels low, especially if the therapeutic agent acts cytotoxically or cytostatically to release virus from the CD4⁺ active and memory cells. Additionally, continuing such treatments protects the patient, who may be severely immunosuppressed or immunocompromised, against opportunistic infection.

At some point during the course of therapy, it may become appropriate to reduce or even cease HAART and administration or coadministration of the inventive therapeutic agents. Generally, the endpoint might preferably occur when the level of active virus is undetectable and the number of CD4⁺ T lymphocyte memory cells, especially those containing HIV, is undetectably low. The level of active virus may be considered undetectably low using conventional assays of viral activity, including measuring copies of HIV RNA/ml (about 50 copies/ml). The number of CD4⁺ T lymphocyte memory cells can likewise be determined using conventional assays and screens.

Of course, if drugs for warding off opportunistic infections are being administered or coadministered, it would not be appropriate to wean a patient from those drugs until the patient's immune system has been appropriately reestablished. Administration or coadministration of HAART and CPT according to the invention will likely result in loss of some acquired immunity, leaving the patient in an immunosuppressed state.

If the patient's immune system does not spontaneously reemerge from its immunosuppressed state after ceasing HAART and the inventive therapy, then it may be necessary to intervene further. This intervention may take the form of reestablishing the patient's immune system through procedures such as bone marrow transplants, thymic stimulation, administration of various cytokine growth factors and/or interleukins, vaccination, and other similar, conventional, procedures. The patient's immune system may be considered reestablished when conventional measures of immune system function have returned to reasonably normal levels.

Reestablishment of the patient's immune system, particularly the CD4⁺ subset, presupposes the existence of stem cells that are relatively resistant to HIV infection and that can be differentiated so as to resupply the patient with CD4⁺ T cells. During ontogeny and in T cell development, precursors of T cells migrate from the bone marrow to the thymus, where most T cell development occurs. In the thymus, T cells mature and express antigen specificity, and are selected for appropriate antigen binding. More complete discussion of T cell development may be found in “Cancer: Principles and Practice of Oncology” (1997) (Vincent DeVita, et al., eds.)

Practicing the invention as disclosed permits these stem cells to undergo the thymic maturation process and develop into mature CD4⁺ cells at a significantly reduced risk of HIV infection. Furthermore, it is within the scope of the invention to stimulate the production of stem cells (through, e.g., bone marrow transplants), and of mature CD4⁺ and other immune system components (through various forms of immunostimulation).

After the patient's immune system has been reasonably reestablished, the patient may be weaned from the drugs that are administered or co-administered to ward off opportunistic infections. During the process of weaning from these drugs, and from HAART and CPT, for that matter, the patient should be closely monitored for signs of relapse. Such signs include increasing active HIV load, abnormal T cell counts, symptoms of opportunistic infections, etc. If signs of relapse are seen, then the patient should not be weaned from their medications for a further evaluation period. It may be necessary to make further adjustments to the patient's therapy, up to and including repeating practice of the present invention to eliminate residual reservoirs of HIV.

If the patient is successfully weaned from the last of the HAART, CPT, and anti-infection drugs to ward off opportunistic infections, and the patient's immune system is stable, then it may be possible for the patient to be in remission for long periods of time. Of course, during that time, the patient should be routinely monitored for reemergent signs of infection. If such signs reemerge, then the patient may require repeat treatments according to the invention.

In another embodiment, the methods of the present invention may be practiced in an in vitro or ex vivo environment. All of the discussion above that is relevant to an in vitro or ex vivo environment applies to such embodiments. In particular, practice of an in vitro or ex vivo embodiment of the invention might be useful in the practice of immune system transplants, such as bone marrow transplants or peripheral stem cell procurement. In such procedures, the inventive therapeutic agents might be used, as generally described above, to purge the transplant material to reduce the risk of HIV infection due to HIV-infected memory T cells.

In another embodiment, practice of the invention might be used to purge whole blood supplies to reduce the risk of HIV infection due to HIV-infected memory T cells. Other applications such in vitro or ex vivo applications will occur to one of skill in the art and are therefore contemplated as being within the scope of the invention.

It will be apparent to those skilled in the art that various modifications and variations can be made in the methods, kits and compositions of the present invention without departing from the spirit or scope of the invention. Thus, it is intended that the present invention cover the modifications and variations of this invention provided they come within the scope of the appended claims and their equivalents.

What is claimed is:

1. A method for treating an HJV-infected host comprising:
treating an HIV-infected host with a pharmaceutically effective amount of one or more agents selected from the group consisting of nucleoside reverse transcriptase inhibitor, non-nucleoside reverse transcriptase inhibitors, protease inhibitor, fusion inhibitor and integrase inhibitor until the host has a low to undetectable plasma HIV-1 viral load which is below 50 copies of active HIV RNA per ml of plasma;
then administering to the HIV-infected host a therapeutically effective amount of a composition comprising a compound selected from the group consisting of 20(S)-camptothecin, analog of 20(S)-camptothecin, derivative of 20(S)-camptothecin, prodrug of 20(S)-camptothecin and pharmaceutically active metabolite of 20(S)-camptothecin; and
then administering to the host an immuno-modulator to reestablish the host's immune system.

2. The method according to claim 1, wherein the composition comprises 9-nitro-20(S)-camptothecin or 9-amino-20(S)-camptothecin.

3. The method according to claim 1, wherein the host is also suffering from Kaposi's sarcoma, Hodgkin's lymphoma, or non-Hodgkin's lymphoma.

4. The method according to claim 1, wherein the nucleoside reverse transcriptase inhibitor is selected from the group consisting of zidovudine, didanosine, zalcitabine, lamivudine, stavudine, abacavir, and adefovir dipivoxil.

5. The method according to claim 1, wherein the protease inhibitor is selected from the group consisting of indinavir, ritonavir, saqinavir, nelfinavir, and amprenavir.

6. The method of claim 1, wherein administering to the HIV-infected host includes administering or coadministering parenterally, intraperitoneally, intravenously, intraartierally, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, and intrathecally.

7. A method of treating an HIV-infected host comprising:
treating an HIV-infected host with highly active antiretroviral therapy (HAART) until the host has a low to undetectable plasma HIV-1 viral load which is below 50 copies of active HIV RNA per ml of plasma;
detecting latent reservoirs of HIV in memory T cells of the HIV-infected host;
administering to the HIV-infected host therapeutically effective amount of a composition comprising a compound selected from the group consisting of 20(S)-camptothecin, analog of 20(S)-camptothecin, derivative of 20(S)-camptothecin, prodrug of 20(S)-camptothecin and pharmaceutically active metabolite of 20(S)-camptothecin; and
then administering to the host an immuno-modulator to reestablish the host's immune system.

8. The method of claim 7, wherein the host is also suffering from Kaposi's sarcoma, Hodgkin's lymphoma, or non-Hodgkin's lymphoma.

9. The method of claim 1, wherein the immuno-modulator is selected from the group consisting of AS-101, BROPIRIMINE, ACEMANNAN, CL246728, EL10, γ-interferon, granulocyte macrophage colony stimulating factor, interleukin-2, α-2-interferon, α-2a-interferon, IMREG-1, IMREG-2, methionine-enkephalin, muramyl-tripeptide granulocyte macrophage colony stimulating factor, rCD4, SK&F106528, and tumor necrosis factor.

10. The method of claim 1, wherein the immuno-modulator is bone marrow transplant.

11. The method of claim 7, wherein the immuno-modulator is selected from the group consisting of AS-101, BROPIRIMINE, ACEMANNAN, CL246728, EL10, γ-interferon, granulocyte macrophage colony stimulating factor, interleukin-2, α-2-interferon, α-2a-interferon, IMREG-1, IMREG-2, methionine-enkephalin, muramyl-tripeptide granulocyte macrophage colony stimulating factor, rCD4, SK&F106528, and tumor necrosis factor.

12. The method of claim 1, wherein the immuno-modulator is bone marrow transplant.
